(19) United States
(12) Patent Application Publication
(10) Pub. No.: US 2024/0122583 A1
(43) Pub. Date: Apr. 18, 2024

(54) A MALE GENITAL SHEDDING CELL SAMPLING DEVICE

(71) Applicant: Hefei Beini Medical Technology Co., LTD, Hefei (CN)

(72) Inventors: WEI WANG, Hefei (CN); ANJIE WANG, Hefei (CN)

(21) Appl. No.: 18/325,092

(22) Filed: May 29, 2023

(30) Foreign Application Priority Data
Oct. 18, 2022 (CN) 202211272570.7

(51) Int. Cl. A61B 10/02

(52) U.S. Cl. CPC A61B 10/02; A61B 2010/0225

(57) ABSTRACT

The present invention relates to the field of male genitalia sampling technology, particularly a male genitalia exfoliated cell sampling device, which is equipped with a first sampling part and a second sampling part. When using this device, the first sampling part is inserted into the male urethral opening, and the exfoliated cells from the mucous membrane near the urethral opening are collected by rotating the first sampling part. After removing the first sampling part from the urethra, the second sampling part is then used to collect epithelial exfoliated cells from multiple sites such as the penis, foreskin, glans, scrotum, and testicles. This device can collect two different types of cell tissues with one sampler, making it simple and convenient to use, comprehensive in sampling, and more accurate and safe for detection.

### CROSS-REFERENCE TO RELATED APPLICATIONS

This application claims the benefit and priority of Chinese patent application No. 202211272570.7, filed on Oct. 18, 2022, disclosure of which is hereby incorporated by reference in its entirety.

### TECHNICAL FIELD

This invention relates to the technical field of male genital sampling devices, and in particular, to a male genital shedding cell sampling device.

### BACKGROUND

When sampling the cell tissue of viruses or other pathogens in the location of the male reproductive organs, it is generally necessary to sample the male genital skin and/or the urethral mucosal tissue separately. Sampling in a single way may not be comprehensive. If both methods are used simultaneously, it will be inconvenient to use the sampler, which will increase the procedure of operation, exposure time, and the chance of iatrogenic infection.

### SUMMARY

The purpose of this invention is to solve the problem that using a single sampling method in the existing technology may not be comprehensive, and if two sampling methods are used at the same time, the sampling device may become inconvenient. Therefore, a male reproductive organ detachment cell sampling device is proposed.

To achieve the above purpose, the following technical solution is adopted:
a male reproductive organ detachment cell sampling device comprises a pole body, with a first sampling part and a second sampling part set on the pole body. The first sampling part and the second sampling part are arranged along the length direction of the pole body. The first sampling part is used to insert into the urethra of the male reproductive organ for sampling of the near urethral mucosal detachment cells, and the second sampling part is used to sample the detached cell tissue on the epithelium of multiple parts of the male reproductive organ.

The first sampling part is set near one end of the pole body.

The hardness of the second sampling part is greater than that of the first sampling part.

The first sampling part and the second sampling part are made of brush or plush with different hardness.

The first and/or second sampling parts are made of cotton, cotton cloth, sponge, polyester fiber, plush, polyamide fiber, nylon material, or polyethylene.

The brush of the first sampling part is spirally distributed on the outer circumference of the pole body, and/or the brush of the second sampling part is spirally distributed on the outer circumference of the pole body.

A positioning part is set on the pole body for limiting the depth of insertion of the first sampling part into the urethra.

A ball head part is provided at one end of the pole body away from the first sampling part and close to the second sampling part.

The first sampling part has a diameter of 2 mm to 14 mm and a length of 4 mm to 20 mm. The second sampling part has a diameter of 3 mm to 20 mm and a length of 5 mm to 30 mm.

The rod section has a limit part that is used to limit the depth of insertion of the first sampling part into the urethra. The limit part has a diameter of 4 mm to 15 mm and a thickness of 0.5 mm to 5 mm.

At one end of the rod section, away from the second sampling part, there is a ball head with a diameter of 1 mm to 6 mm.

The cross-section of the rod section is square, hexagonal or octagonal, and the outer diameter of the circumscribed circle of the rod section is 3 mm to 8 mm. The surface of the rod section has a friction pattern.

The rod section has a breakable part that is used to separate the main body part from the part with the first and second sampling parts. The limit part is made of polymer material.

Along the length direction of the rod section is defined as the length dimension, and along the width direction of the rod section is defined as the width dimension. The first sampling part is a spiral conical structure or a laminated conical structure. The end of the first sampling part with a smaller width dimension is close to one end of the rod section, and the end of the first sampling part with a larger width dimension is close to the end of the second sampling part.

The second sampling part is a spiral cylindrical structure or a laminated cylindrical structure, with a width dimension greater than the maximum width dimension of the first sampling part.

The first sampling part is made of cotton, cotton cloth, sponge, polyester fiber, plant velvet, polyamide fiber, nylon material or polyethylene.

The second sampling part and/or is made of cotton, cotton cloth, sponge, polyester fiber, plant velvet, polyamide fiber, nylon material or polyethylene.

The handle part of the pole section has a breaking part, which can separate the handle part from the detection part.

The present invention proposes a male genital shedding cell sampling device. The advantageous effect is that when using this device, the first sampling part is inserted into the male genital urethral opening to collect shedding cells or mucosal cells near the urethral opening through the first sampling part. After the first sampling part is pulled out of the urethra, the second sampling part can be used to collect shedding cells of epithelial tissue from multiple parts such as the penis, foreskin, glans, scrotum, and testicles. Two different types of cell tissues can be collected through one sampler, making it simple, convenient, comprehensive, and precise for detection.

### BRIEF DESCRIPTION OF DRAWINGS

FIG. 1 shows the schematic diagram of the structure of the present invention;

FIG. 2 shows the structural diagram of the sampling part of the spiral structure of the present invention.

FIG. 3 is a schematic diagram of the first sampling part of the present invention with a conical head structure.

FIG. 4 is a schematic diagram of the first sampling part of the present invention with an elliptical spherical structure.

In the figures: the ball head 1, the first sampling part 2, the limiting part 3, the second sampling part 4, the rod body part 5, and the breaking part 6.

### DETAILED DESCRIPTION

This is a description of the technical solution in the embodiments of the present invention, in conjunction with the drawings of the embodiments of the present invention. It is obvious that the described embodiments are only a part of the embodiments of the present invention, not all of them.

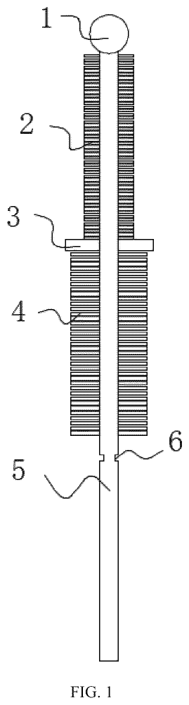

Referring to FIG. 1, a male genital cell sampling device is provided, including a rod body part 5, a first sampling part 2, and a second sampling part 4. The rod body part 5 serves as a carrier for the first sampling part 2 and the second sampling part 4, and can be a plastic rod, a metal rod, or a metal wire braided rod body. The first sampling part 2 and the second sampling part 4 are set along the length direction of the rod body part 5. The first sampling part 2 is used for sampling the mucosal cells detached near the urethral orifice by inserting it into the male urethral orifice, while the second sampling part 4 is used for sampling the epithelial cells detached from multiple sites of the male external genitalia.

When using this device, the first sampling part 2 is inserted into the male urethral orifice for about 0.8 cm to 1.5 cm. The first sampling part 2 collects the detached cells or mucosal cells inside the urethral orifice by rotating it. After the first sampling part 2 is pulled out of the urethra, the second sampling part 4 is used to collect the epithelial cells detached from multiple sites such as the penis, foreskin, glans, scrotum, and testes. Two different types of cell tissue can be collected by a single sampler, which is simple and convenient to use, and the sampling is comprehensive, making the detection more accurate.

As one embodiment, the first sampling part 2 is located near one end of the rod body part 5, which facilitates the insertion of the first sampling part 2 into the urethral orifice.

The first sampling part 2 is used to insert into the urethral opening and sample the mucosal tissue near the urethral opening, while the second sampling part 4 is used to sample the epithelial cells from multiple sites such as the penis, foreskin, glans, scrotum, and testicles. In order to reduce damage to the male genitalia caused by the sampling parts while ensuring sampling effectiveness, the hardness of the second sampling part 4 is greater than that of the first sampling part 2.

The first sampling part 2 and the second sampling part 4 are made of bristles of different hardness, for example, the first sampling part 2 uses soft bristles while the second sampling part 4 uses relatively harder bristles compared to the first sampling part 2. Of course, the first sampling part 2 and/or the second sampling part can be made of materials such as cotton, cotton cloth, sponge, polyester fiber, plush, polyamide fiber, nylon, or polyethylene. It is preferable to make the bristles in the form of a brush.

The bristles of the first sampling part 2 are spirally distributed on the outer peripheral surface of the rod body part 5. This allows the mucosal cell tissue to better adhere to the first sampling part 2 by rotating the rod body part 5 after inserting it into the urethral opening, while the spiral shape of the first sampling part 2 can also reduce damage to the urethra. Similarly, the bristles of the second sampling part 4 may also be spirally distributed on the outer peripheral surface of the rod body part 5. Of course, the first sampling part 2 and/or the second sampling part 4 of the device may also be designed in a multi-row brush overlay manner, although this will cause more damage to the urethra when applied to the first sampling part 2 and will result in poor sampling effectiveness.

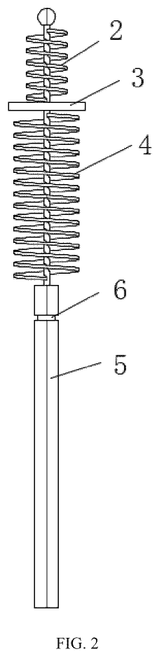

Referring to FIG. 2, this device uses spiral bristles for sampling, which not only facilitates the capture of detached cells, but also allows the detached cells captured by the sampling parts to be better released for subsequent experimental processing. The spiral bristles in FIG. 2 are composed of fine hair bodies in a spiral structure.

As one embodiment, there is a positioning part 3 on the rod body part 5 to limit the depth to which the first sampling part 2 is inserted into the urethra. Since the first sampling part 2 is inserted into the urethra, inserting it too deeply can damage the urethra. By setting the positioning part 3 to prevent the first sampling part 2 from being inserted too deeply, the positioning part 3 can be a disk-shaped plate with a diameter larger than the diameter of the urethral opening, and can be made of materials such as rubber or plastic.

For example, the positioning part 3 is located between the first sampling part 2 and the second sampling part 4. The survival of HPV virus depends on human squamous epithelial cells. The deepest part of the male urethral opening is the fossa navicularis, which is generally located 1.5-2 cm away from the urethral opening. Therefore, the depth of male urethral warts is limited to this depth and will not go further inside. During clinical sampling of the urethra, sampling too deeply can increase the patient's discomfort and bleeding, and the positioning part directly controls the operator's improper operation.

Refer to FIG. 1 or FIG. 2, the first sampling part 2 has a diameter of 2 mm-14 mm and a length of 4 mm-20 mm;

The second sampling part 4 has a diameter of 3 mm-20 mm and a length of 5mm-30mm;

The shaft part 5 is equipped with a limiting part 3, which is used to limit the depth of insertion of the first sampling part 2 into the urethra. The diameter of the limiting part 3 is 4 mm-15 mm, and the thickness is 0.5 mm-5 mm. The shaft part 5 is located at one end away from the second sampling part 4, with a ball head part 1, and the diameter of the ball head part 1 is 1 mm-6 mm.

The cross-section of the shaft part 5 is square, hexagonal, or octagonal, and the outer diameter of the shaft part 5 is 3 mm-8 mm. The surface of the shaft part 5 has friction patterns, which are used to increase the friction when wearing gloves for sampling, control the number of rotations and speed of rotation during sampling, and facilitate medical staff to control the rotation and force of sampling under the principle of aseptic operation for mucosal sampling in the urethra.

The shaft 5 has a broken part 6, and the broken part 6 is used to separate the shaft 5 with the first sampling part 2 and the second sampling part 4 from the main part of the shaft 5.

The limiting part 3 is made of polymer materials such as PE, PET, silicone, etc. In order to avoid bacterial contamination of the first sampling part 2 and the second sampling part 4 by hands, the shaft part 5 usually has an extra length as a handle. The shaft part 5 has a breaking part 6, which is used to separate the shaft part 5 with the first sampling part 2 and the second sampling part 4 from the main body of the shaft part 5. After sampling is completed, the shaft part 5 is broken at the breaking part 6, separating the handle from the first sampling part 2 and the second sampling part 4, and then the sample is tested.

As an embodiment:

A male reproductive organ detachment cell sampling device includes a shaft portion 5 having a first sampling portion 2 and a second sampling portion 4. The shaft portion 5 serves as a carrier for the first sampling portion 2 and the second sampling portion 4, and can be made of plastic, metal or a woven metal wire. The first sampling portion 2 and the second sampling portion 4 are arranged along the length direction of the shaft portion 5. The first sampling portion 2 is used to insert into the male urethra for mucosal cell sampling, while the second sampling portion 4 is used to sample the epithelial cells on multiple parts of the male reproductive organ.

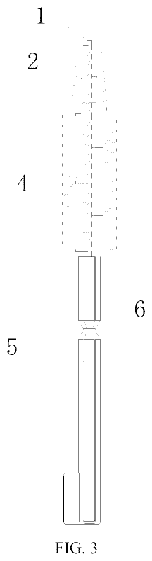

Referring to FIG. 3, the length direction of the shaft portion 5 is defined as the length dimension, and the width direction of the shaft portion 5 is defined as the width dimension. The first sampling portion 2 is in a spiral cone structure, or a laminated cone structure. The spiral cone structure refers to the overall shape of the first sampling portion 2 being a cone structure and being in a spiral structure. The laminated cone structure refers to the overall shape of the first sampling portion 2 being a cone structure and being formed by a series of interlocking circular disks with different diameters. The smaller end of the first sampling portion 2 in the width dimension is close to one end of the shaft portion 5, while the larger end of the first sampling portion 2 in the width dimension is close to one end of the second sampling portion 4.

The second sampling portion 4 is in a spiral cylindrical structure or a laminated cylindrical structure. The spiral cylindrical structure refers to the overall shape of the second sampling portion 4 being cylindrical and being in a spiral structure. The laminated cylindrical structure refers to the overall shape of the second sampling portion 4 being cylindrical and being formed by a series of interlocking circular disks with the same diameter. The width dimension of the second sampling portion 4 is greater than the maximum width dimension of the first sampling portion 2. The second sampling portion 4 serves as a limit to prevent the first sampling portion 2 from being inserted too deeply and causing damage to the urethra.

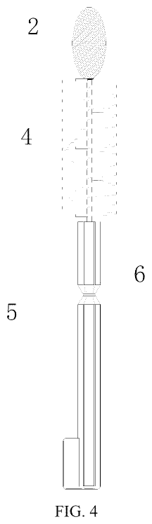

As an implementation method, as shown in FIG. 4, the first sampling part 2 is an elliptical spherical structure, and the second sampling part 4 is either a spiral cylindrical structure or a laminated cylindrical structure, with a width size greater than the maximum width size of the first sampling part 2.

The first sampling part 2 is made of cotton, cotton cloth, sponge, polyester fiber, plant velvet, polyamide fiber, nylon material, or polyethylene; and/or the second sampling part 4 is made of cotton, cotton cloth, sponge, polyester fiber, plant velvet, polyamide fiber, nylon material, or polyethylene.

As an implementation method, when the first sampling part 2 is cone-shaped and the second sampling part 4 is cylindrical, both the first sampling part 2 and the second sampling part 4 are made of brushed material, and are formed into a laminated structure by brushing into a spiral or circular shape. To protect the urethra, the rod body part 5 is provided with a ball head part 1 at one end of the first sampling part 2, away from the second sampling part 4, with a diameter of 1 mm-6 mm.

As another implementation method, when the first sampling part 2 is an elliptical spherical structure, its material is cotton, cotton cloth, sponge, plant velvet, or polyamide fiber.

In the above implementation methods, the cross-section of the hand-held part of the rod body part 5 is square, hexagonal, or octagonal, with an outer diameter of 3 mm-8 mm; the surface of the hand-held part of the rod body part 5 has a friction pattern; and the hand-held part of the rod body part 5 has a breakable part 6, which can separate the hand-held part from the detection part.

The above-described implementation methods are only the preferred embodiments of the present invention, but the protection scope of the present invention is not limited thereto. Any person skilled in the art within the technical scope disclosed in the present invention can obtain a technical solution, idea, or design equivalent to or modified from the technical scheme and invention conception of the present invention, which should be encompassed within the protection scope of the present invention.

What is claimed is:

1. A male genital shedding cell sampling device, comprising a rod portion (5), wherein the rod portion (5) is provided with a first sampling portion (2) and a second sampling portion (4) along the length direction of the rod portion (5), the first sampling portion (2) is used for inserting into the male genital urethral orifice to sample the shedding cells of the mucosa near the urethral orifice, and the second sampling portion (4) is used for sampling the shedding cells of the epithelial tissue on multiple parts of the male genital.

2. The male genital shedding cell sampling device according to claim 1, wherein the first sampling portion (2) is located at one end near the rod portion (5), and the second sampling portion (4) is located at the other end of the first sampling portion (2).

3. The male genital shedding cell sampling device according to claim 2, wherein the hardness of the second sampling portion (4) is greater than that of the first sampling portion (2).

4. The male genital shedding cell sampling device according to claim 3, wherein the first sampling portion (2) and the second sampling portion (4) are made of different hardness of bristles or velvets.

5. The male genital shedding cell sampling device according to claim 2, wherein the first sampling portion (2) is made of cotton, cotton cloth, sponge, polyester fiber, velvet, polyamide fiber, nylon material, or polyethylene, and/or the second sampling portion (4) is made of cotton, cotton cloth, sponge, polyester fiber, velvet, polyamide fiber, nylon material, or polyethylene.

6. A male reproductive cell sampling device according to claim 3, characterized in that the bristles of the first sampling portion (2) are spirally distributed on the outer peripheral surface of the stein portion (5); and/or the bristles of the second sampling portion (4) are spirally distributed on the outer peripheral surface of the stein portion (5).

7. A male genitalia exfoliated cell sampling device according to claim 1, characterized in that a limiting part (3) is provided on the rod body part (5), the limiting part (3) is used to limit the depth of insertion of the first sampling part (2) into the urethra.

8. A male reproductive cell sampling device according to claim 7, characterized in that the limited position portion (3) is located between the first sampling portion (2) and the second sampling portion (4).

9. A male genitalia exfoliated cell sampling device according to claim 1, characterized in that the first sampling part (2) has a diameter of 2 mm-14 mm and a length of 4 mm-20 mm; the second sampling part (4) has a diameter of 3 mm-20 mm and a length of 5 mm-30 mm. A limited position portion (3) is provided on the stein portion (5), and the limited position portion (3) is used to limit the depth of insertion of the first sampling portion (2) into the urethra, and the diameter of the limited position portion (3) is 4 mm-15 mm, and the thickness is 0.5 mm-5 mm;
The stein portion (5) is provided with a ball head portion (1) at one end far from the second sampling portion (4), and the diameter of the ball head portion (1) is 1 mm-6 mm;
The cross-sectional shape of the stein portion (5) is square, hexagonal or octagonal, and the outer diameter of the circumscribed circle of the stein portion (5) is 3 mm-8 mm;
The surface of the stein portion (5) has frictional patterns; the stein portion (5) has a breaking portion (6), and the breaking portion (6) is used to detach the stein portion (5) with the first sampling portion (2) and the second sampling portion (4) from the main portion of the stein portion (5);
The limited position portion (3) is made of polymer material.

10. A male reproductive cell sampling device according to claim 3, characterized in that the first sampling portion (2) is a spiral cone-shaped structure, or the first sampling portion (2) is a laminated cone-shaped structure, and the smaller width of the first sampling portion (2) is close to one end of the stein portion (5), and the larger width of the first sampling portion (2) is close to the end of the second sampling portion (4).

11. A male reproductive organ shedding cell sampling device according to claim 10, characterized in that the second sampling part (4) is a spiral cylindrical structure, or the second sampling part (4) is a stacked cylindrical structure, and the width dimension of the second sampling part (4) is greater than the maximum width dimension of the first sampling part (2).

12. A male reproductive organ shedding cell sampling device according to claim 3, characterized in that the first sampling part (2) is an elliptical spherical structure, the second sampling part (4) is a spiral cylindrical structure, or the second sampling part (4) is a stacked cylindrical structure, and the width dimension of the second sampling part (4) is greater than the maximum width dimension of the first sampling part (2).

13. A male genitalia exfoliated cell sampling device according to claim 10, characterized in that the first sampling part (2) is made of cotton, cotton cloth, sponge, polyester fiber, flocking, polyamide fiber, nylon material, or polyethylene; and/or the second sampling part (4) is made of cotton, cotton cloth, sponge, polyester fiber, flocking, polyamide fiber, nylon material, or polyethylene.

14. A male genitalia exfoliated cell sampling device according to claim 1, characterized in that the rod body part (5) is provided with a ball head part (1) at one end of the first sampling part (2) away from the second sampling part (4), the ball head part (1) having a diameter of 1 mm-6 mm.

15. A male genitalia exfoliated cell sampling device according to claim 10, characterized in that the first sampling part (2) has a maximum width dimension of 2 mm-14 mm and a length dimension of 4 mm-20 mm.

16. The width dimension of the second sampling part (4) is 3 mm-20 mm and the length dimension is 5 mm-30 mm; the rod part (5) has a detection part and a hand-held part, and the first sampling part (2) and the second sampling part (4) are both located in the detection part;
The cross-section of the hand-held part of the rod part (5) is square, hexagonal, or octagonal, the outer diameter of the circumscribed circle of the hand-held part of the rod part (5) is 3 mm-8 mm, and the surface of the hand-held part of the rod part (5) has a friction pattern;
The hand-held part of the rod part (5) has a breaking part (6), and the breaking part (6) can disconnect the hand-held part from the detection part.
